(19) United States
(12) Patent Application Publication
(10) Pub. No.: US 2024/0416063 A1
(43) Pub. Date: Dec. 19, 2024

(54) Apparatus for Assisting Infant Respiration

(71) Applicant: Gregory G. Faimon, Valley Center, KS (US)

(72) Inventor: Gregory G. Faimon, Valley Center, KS (US)

(21) Appl. No.: 18/816,147

(22) Filed: Aug. 27, 2024

(51) Int. Cl. A61M 16/06; A61M 16/00

(52) U.S. Cl. CPC A61M 16/0605; A61M 16/021

(57) ABSTRACT

An apparatus for assisting infant respiration incorporating a segmented case having a forward segment, a rearward segment, and a seam between the segments; segment mounting screw threads connected operatively to the forward and rearward segments, the screw threads being adapted for releasably positioning ends of the forward and rearward segments at the seam; output and intake ports respectively opening the case at the forward and rearward segments; a respirator mask; a coupling hose operatively connecting the respirator mask to the air output port; a fan and electric motor combination mounted within the rearward segment, the fan and electric motor combination being adapted for drawing air into the intake port, and for driving the air toward the forward segment; and water-based air-cooling media within the forward segment.

### FIELD OF THE INVENTION

The instant invention relates to mechanical respiratory ventilators. More particularly, the instant invention relates to such mechanical ventilators which incorporate adaptations for simultaneously cooling and humidifying ventilation air.

### BACKGROUND OF THE INVENTION

Known mechanisms, apparatus, and assemblies for providing continuous positive airway pressure (CPAP) to an infant are commonly mechanically complex, are cumbersome in assembly and use, and include a multiplicity of components and parts which may not be economically produced and provided. Where such apparatus and assemblies include adaptations for cooling and/or humidifying the provided respiration air, such apparatus and assemblies are commonly further mechanically complex and are less economically assembled and provided.

The instant inventive apparatus for assisting infant respiration solves or ameliorates the above-described deficiencies of prior art apparatus by providing a specialized segmented case which incorporates forward and rearward structural features respectively dedicated to humidity enhanced air-cooling, and to air driving.

### BRIEF SUMMARY OF THE INVENTION

A first structural component of the instant inventive apparatus for assisting infant respiration comprises a segmented case which forwardly houses and operatively supports air cooling and humidifying components as described below, and which rearwardly houses and supports air driving components as described below. In a preferred embodiment, the segmented case comprises a hollow forward segment, a hollow rearward segment, and a seam situated at a releasable juncture between such segments. In a suitable embodiment, the case has rectangular left, right, upper, and lower walls which are arranged in a box configuration. Alternatively, the case may be substantially cylindrical. In each embodiment, the case is preferably composed of durable plastic.

Further structural components of the instant inventive apparatus comprise segment mounting means which are connected operatively to the forward and rearward segments, such connections being situated at the seam. In the preferred embodiment, the segment mounting means are adapted for releasably attaching a rearward end of the case's forward segment to a forward end of the case's rearward segment. In suitable embodiments, the segment mounting means may comprise a fastener selected from nesting flanges, pressure fitted joints, helically threaded fasteners, and releasable latches.

Further structural components of the instant inventive apparatus comprise air output and air intake ports which respectively open the case at its forward and rearward ends. In a preferred embodiment, the apparatus's air intake port opens the case at a rearward end of the rear segment, and the case's air output port opens the case at a forward end of the forward segment.

Further structural components of the instant inventive apparatus comprise a respirator mask, in combination with respirator mounting means which provide for communication of the respirator mask with the case's air output port. In a preferred embodiment, the respirator mask is form-fitted for comfortably covering an infant's nose and mouth. The respirator mask mounting means component may incorporate releasable couplers and a flexible hose or tube which extends from the case to the mask

A further structural component of the instant inventive apparatus comprises a fan and electric motor combination which is housed and supported within the case's rearward segment. In the preferred embodiment, the fan and motor combination is adapted for drawing ambient outside air into the case through the case's air intake port, and for simultaneously driving the air forwardly through the case for emission out of the forward segment's air output port. The fan driven air communicates with the respirator mask via the respirator mask mounting means to provide positive airway pressure to an infant wearing the mask.

A further component of the instant inventive apparatus comprises water-based air-cooling media which are received and housed within the case's forward segment. In a suitable embodiment, the water-based air-cooling media comprise a body of broken or cubed ice. Alternatively, the water-based air-cooling media may comprise an hydrophilic material through which air may pass such as excelsior fill, poly-fil, fabric wadding, or open celled elastomeric foam. Where the water-based air-cooling media comprise such hydrophilic material, water is preferably introduced into and absorbed within the material prior to or simultaneously with the material's receipt within the case's forward segment.

In operation of the instant invention, electric actuation of the apparatus' fan and electric motor combination draws ambient outside air into and through the case. During the forward passage of the air through the case, the air courses over and through the water-based air-cooling media which simultaneously cools and humidifies the air. The water-based air-cooling media facilitates evaporative cooling of the air and, where the media comprises ice, the air is additionally conductively cooled. Following these cooling and humidifying steps, the air emits from the case's output port to flow to the attached respirator mask.

The inventive apparatus is capable of supplying beneficially treated air to an infant suffering from bronchial congestion and inflammation due to cold or croup. The treated air may be advantageously maintained by the apparatus at an appropriate infant CPAP pressure between 0.007 psi and 0.07 psi (or between 0.5 cm and 5 cm of water) above ambient air pressure. The cooled nature of the air which is provided by the apparatus tends to sooth or avoid irritation of the infant's oropharynx, larynx and bronchial passages. The enhanced air humidity provided by the apparatus simultaneously avoids the irritating airway drying which is commonly associated with breathing cold air.

Accordingly, objects of the instant invention include the provision of an apparatus for assisting infant respiration which incorporates structures as described above, and which arranges those structures in relation to each other in manners described above.

Other and further objects, benefits, and advantages of the instant invention will become known to those skilled in the art upon review of the detailed description which follows, and upon review of the appended drawings.

### BRIEF DESCRIPTION OF DRAWINGS

FIG. 1 is a perspective view of a suitable embodiment of the instant inventive apparatus for assisting infant respiration.

FIG. 2 is a sectional view as indicated in FIG. 1.

FIG. 3 re-depicts the structure of FIG. 2, the view of FIG. 3 showing an added air-cooling media component, and showing components in a partially exploded view.

FIG. 4 presents an alternate configuration of the structures depicted in FIG. 2.

FIG. 5 presents an alternate configuration of the apparatus of FIG. 1.

FIG. 6 is a sectional view as indicated in FIG. 5.

FIG. 7 is a partially exploded view of structures depicted in FIG. 6.

FIG. 8 re-depicts the structure of FIG. 6, the view of FIG. 8 showing ice cubes within an air-cooling media receiving space.

### DETAILED DESCRIPTION OF PREFERRED EMBODIMENTS

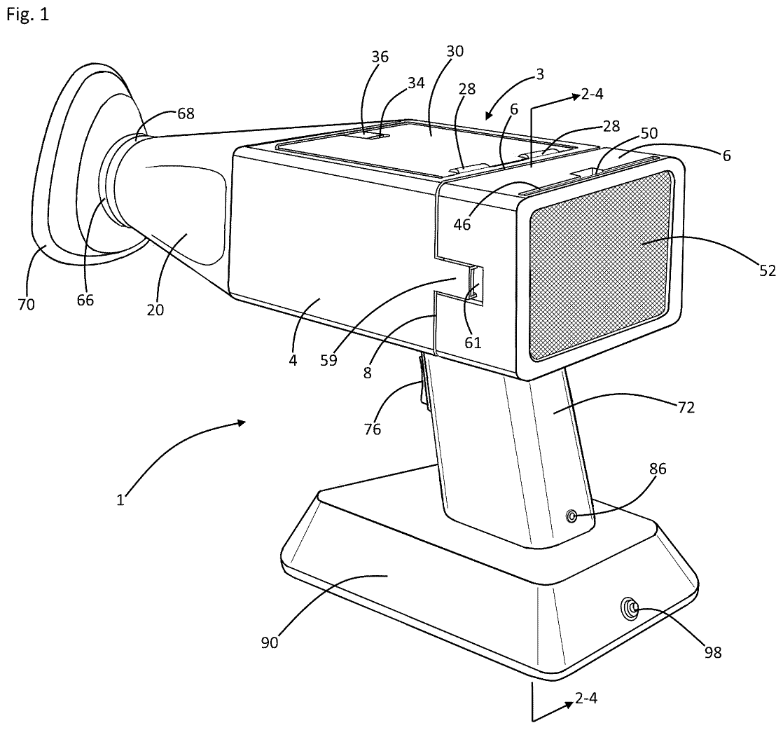

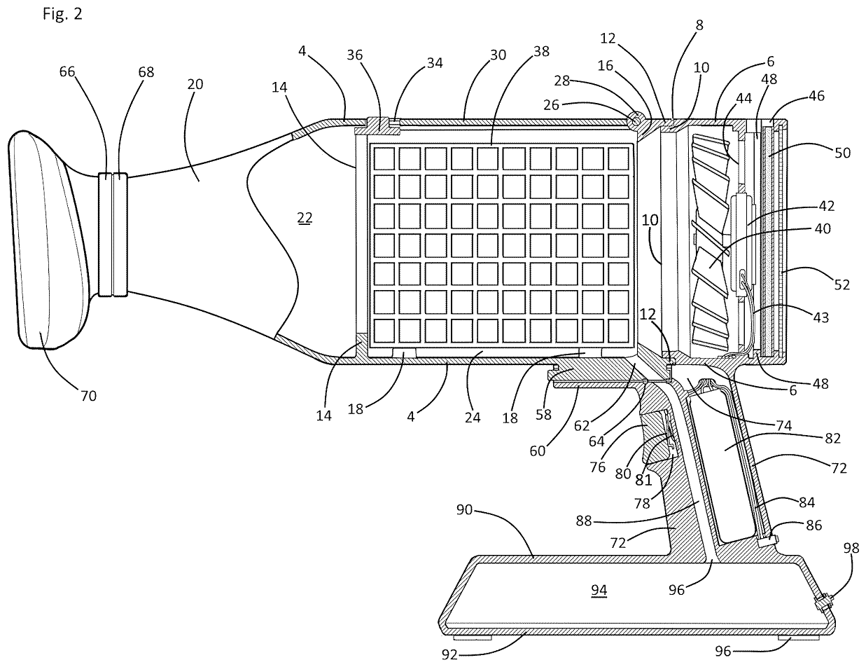

Referring now to the drawings, and in particular to FIGS. 1 and 2, a suitable embodiment of the instant inventive apparatus for assisting infant respiration is referred to generally by reference arrow 1. The apparatus 1 preferably comprises a case which is referred to generally by reference arrow 3, the case 3 preferably comprising a hollow forward segment 4 and a hollow rearward segment 6, such segments joining at a seam 8. The forward end of the rearward segment 6 suitably forms an inner stepped flange 10 whose outer periphery is fitted for slidably receiving the inner periphery of an outer flange 12, such fitted flanges 10 and 12 form a slidably attachable and releasable joint. Slide fittings 58 and 60 at the undersurface of the case 3 receive and guide the forward segment 4 into alignment with the rearward segments, and left and right releasable latch fasteners 59,61 secure and releasably hold the forward segment 4 upon and in communication with the rearward segment 6.

The case's forward segment 4 preferably opens forwardly at flange 14 for communication of its hollow interior 24 with the hollow interior 22 of a forward conic case segment 20. An infant's or child's respiration facemask 70 is suitably directly fixedly attached to the forward opening of the conic section 20. In the FIG. 2 embodiment, releasable couplings 66, 68 are suitably provided for attaching the mask 70 to the cone section 20, and for facilitating detachment for separate mask cleaning and sanitization.

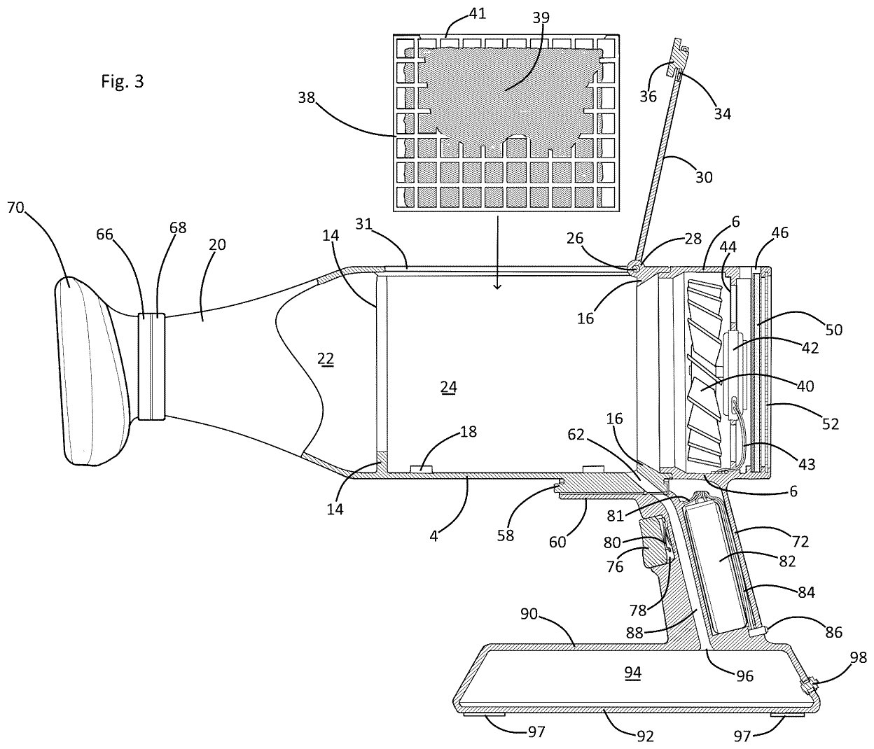

Referring further simultaneously to FIG. 3, a perforate or open framed basket 38 having an upper opening 41 may be downwardly received through an upper port 31 into the interior 24 of the forward segment 4. The walls of the basket 38 suitably comprise open grids which facilitate air flow into and through the basket. Water-based air-cooling media, such as a body of hydrophilic open cell elastomeric foam 39, may be downwardly received 1 into the interior space of the basket 38 through upper opening 4. Thereafter, such basket 38 may be downwardly inserted through port 31 into space 24.

The front output port forming flange 14 in combination with a rear flange 16 secures the basket 38 against forward and rearward movement within space 24, and upon closure of lid 38 via pivoting motion about hinge 26, 28, and upon latching of the lid's slide latch 34, 36, the basket 38 and air-cooling media 39 are captured and retained within the front segment's space 24. Riser flanges 18 extend upwardly from the floor of the front segment 4, such flanges separating the floor of the basket 38 from the floor of the forward segment 4 to facilitate water drainage. A drain channel 62 opens at the rearward end of the floor of the forward segment 4 to further facilitate water drainage.

A fan 40 and electric motor 42 combination is operatively housed and supported within the case's rearward segment 6 by motor support arms 44. The rearward end of such segment 6 is preferably opened by an air intake port which is preferably being covered by a debris filtering screen 52. A high efficiency particulate air filter or HEPA filter 50 is preferably slidably and removably received within the case's rearward segment 6, such filter suitably residing between the fan and motor combination 40, 42. The HIPA filter 50 may be slidably inserted into and removed through slot 46, the inserted filter being retained by a peripherally extending flange 48.

A handle 72 having a hollow interior 74 is preferably fixedly attached to and extends downwardly from the case's rearward segment 6. In a preferred embodiment, a hollow interior 74 of the handle 72 supports and houses a rechargeable lithium-ion battery 82. Electric motor powering conductors 43 extend from the battery 82 to the electric motor 42 for powering such motor, and a charging port 86 and electrically attached recharging conductors 84 facilitate recharging of the battery 82. An on/off switch 80 adapted for alternatively completing and breaking an electric power circuit to motor 42 via conductors 81 is preferably provided, such on/off switch 80 being actuatable by a trigger pull 76 which slidably moves within a slide channel space 78, formed within the handle's forward wall.

A support base 90 is preferably fixedly attached to or formed wholly with the lower end of the handle 72, such base preferably forming and defining an interior water collection reservoir 94. A fluid passage conduit 88 preferably extends through the handle 72, the upper end of such conduit 88 communicating with the forward segment's drain channel 62, and the lower end of such channel 88 opening at a second port 96 in communication with the water collection reservoir 94. Friction pads 97 are preferably fixedly attached to the undersurface of the lower floor 92 of the base 90. A drain port and removable plug 98 combination is preferably provided to facilitate drainage of the water collection reservoir 94.

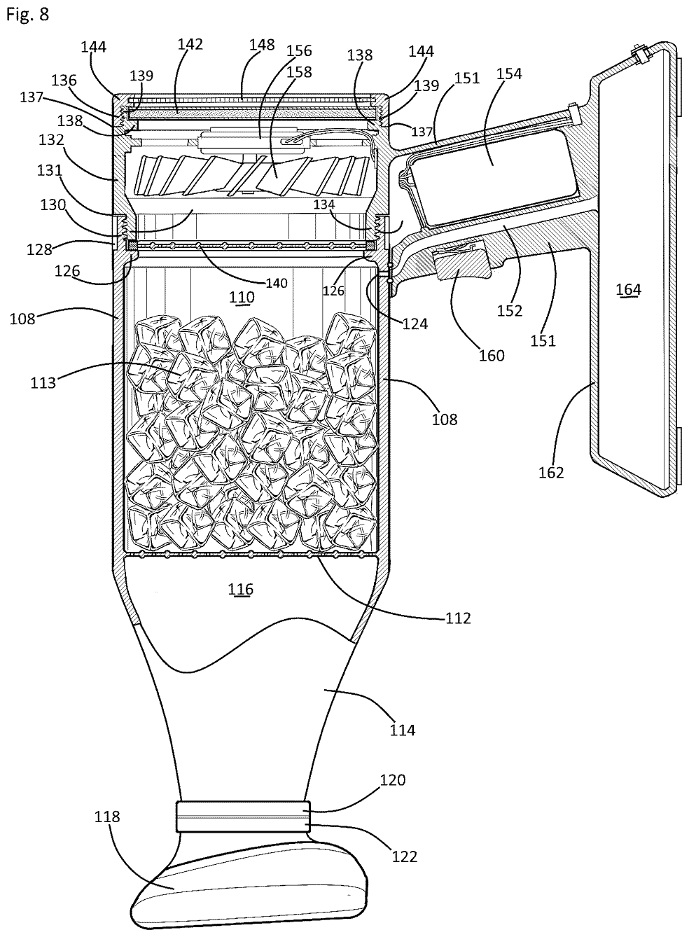

In operation of the inventive apparatus for assisting infant respiration 1, a caretaker of an infant in need of respiration assistance may initially hold basket 38 while inserting a body of hydrophilic material downwardly through the basket's upper opening 41. Thereafter, the caretaker may pour clean water into the material 39, allowing the material to absorb and retain the water. The hydrophilic material constitutes a water-based air-cooling media component of the inventive apparatus, such material suitably comprising a body of open cell elastomeric foam 39. Such media component may suitably alternatively comprise woven fabric wadding such as strips of terrycloth or wash cloth material. Alternatively, a body of excelsior may be inserted into the basket's interior space. As indicated in FIG. 8, the water-based air-cooling media may comprise cubed or crushed ice 113 which may be alternatively received within the basket 38.

Thereafter, the infant's caretaker may downwardly insert the basket 38 and water-based media 39 through the case's opened port 31 until the lower floor of the basket 38 rests upon upper surfaces of risers 18. Thereafter, the caretaker may pivotally move lid 30 about hinges 26, 28, and may operate slide latch 39,36 to secure the lid 30 at its closed position as indicated in FIG. 2. Following closure of the lid, the basket 38 and water-based media 39 are securely received and held within space 24.

Thereafter, the caretaker may place mask 70 over the nose and mouth of the infant while rearwardly depressing the on/off switch trigger 76. Such trigger depression closes switch 80 and actuates the motor and fan 42, 40 via electric power supplied by the handle mounted lithium-ion battery 82. In a suitable embodiment, the rechargeable battery 82 may be eliminated, and the motor 42 may be powered directly by a power cord and wall socket plug.

In a preferred embodiment, the motor 42 and fan 40 are adapted to produce air pressure within the interior of the case 3 between 0.5 cm and 5 cm of water above ambient atmosphere pressure (i.e. between 0.007 psi and 0.07 psi above ambient atmospheric pressure), such air pressure range being compatible with and assistive of infant respiration. As the pressurized air flows over and through the water-based air-cooling media 39, the air is simultaneously cooled and humidified. Where the infant suffers from bronchial congestion and inflammation, the cooled air soothes and relieves irritation, while the air's enhanced humidity ameliorates the typical airway drying effects of breathing cold air.

During use of the instant inventive apparatus, unevaporated water which may flow downwardly from media 39 may advantageously drain and escape rearwardly between risers 18 and downwardly through channel 62. Such water further drains downwardly via handle channel 88 to emit at a second or underlying drain port 96 into a water reservoir 90,94. Upon filling of the reservoir, the caretaker may remove a provided drain plug 98 and may drain the reservoir 94. The expanded volume of the reservoir 94 advantageously allows the reservoir to additionally and simultaneously function as a support base 90.

Following the infant's use of the instant inventive apparatus 1, the caretaker may disengage left and right latches 59, 61 and may forwardly slidably remove the case's forward segment 4 from the case's rearward segment 6. Thereafter, the operator may open lid 30 by slidably disengaging latch 34, 36. Thereafter, the operator may upwardly extract the basket 38 through opened port 31. Thereafter, the operator may extract the cooling media 39 through the upper opening 41 of the basket 38. Thereafter, coupler halves 66 and 68 may be disengaged, allowing the facemask 70 to be separated from the forward segment's conic section 20. Following such disassembly steps, each of the removed components may be sanitized with a disinfectant. Thereafter, the sanitized components of the apparatus may be re-assembled to the configuration depicted in FIG. 1.

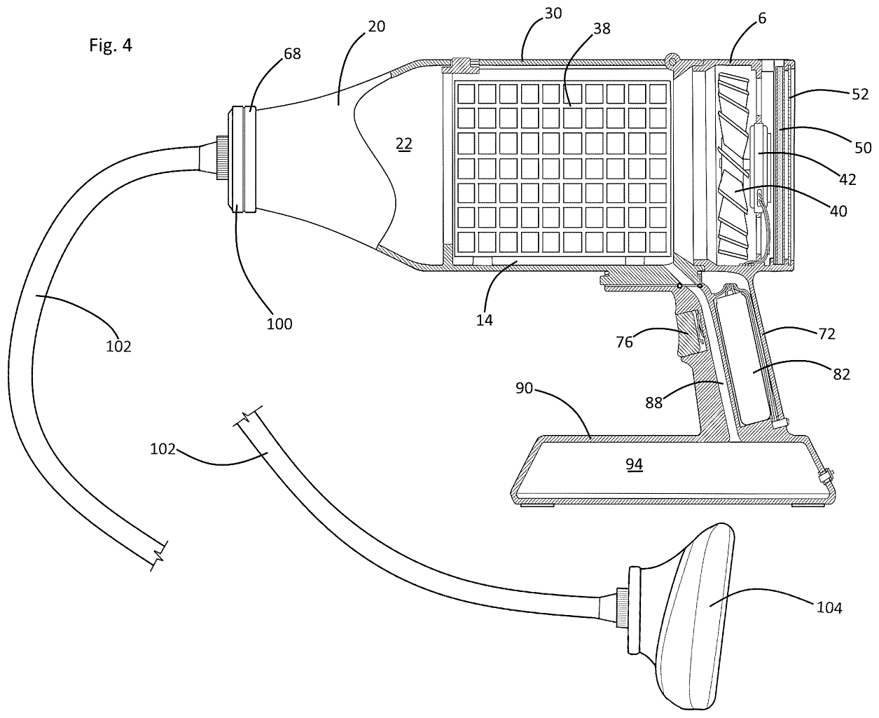

In the alternative configuration of FIG. 4, the mask 70 and coupler half 66 of the FIG. 1 configuration are removed, and an alternative mask 104 having a hose extension 102 is mounted in its place. In the FIG. 4 configuration, coupler half 100 is substantially identical to coupler half 66 of the FIG. 1 configuration, such coupler half 100 being removably attachable similarly with coupler half 66. In the FIG. 1 configuration, the assembly's respirator mask mounting means comprise directly interconnected coupler halves 66 and 68, and in the FIG. 4 alternative such mounting means further comprise hose extension 102. In use of the apparatus of FIG. 4, the base 90 may advantageously rest upon a surface such as a bedside tabletop while mask 108 and extension hose 102 are extended to the infant's nose and mouth. Friction pads 97 attached to the bottom of base 90 assist in securing the apparatus upon such surface during such use.

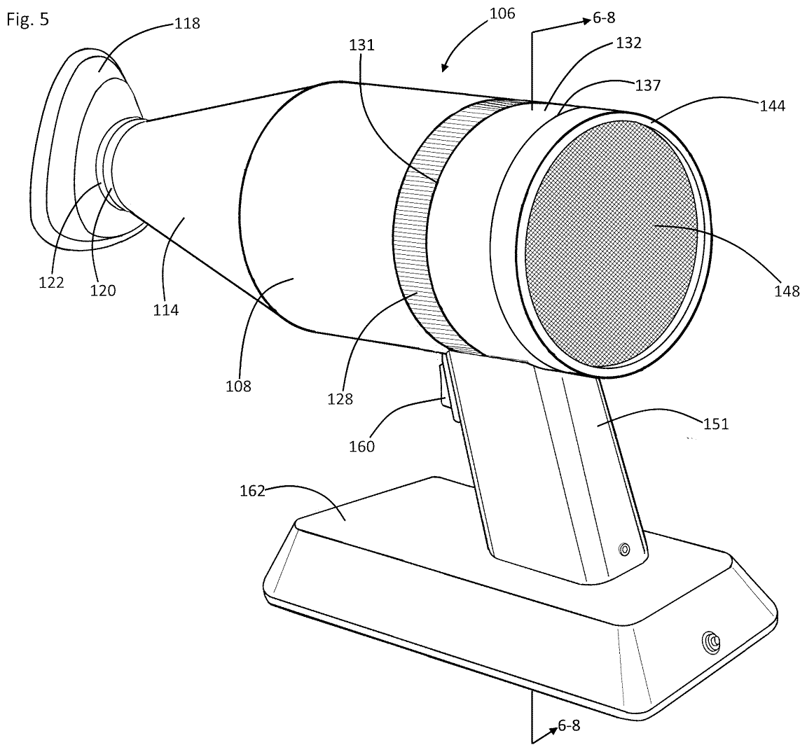

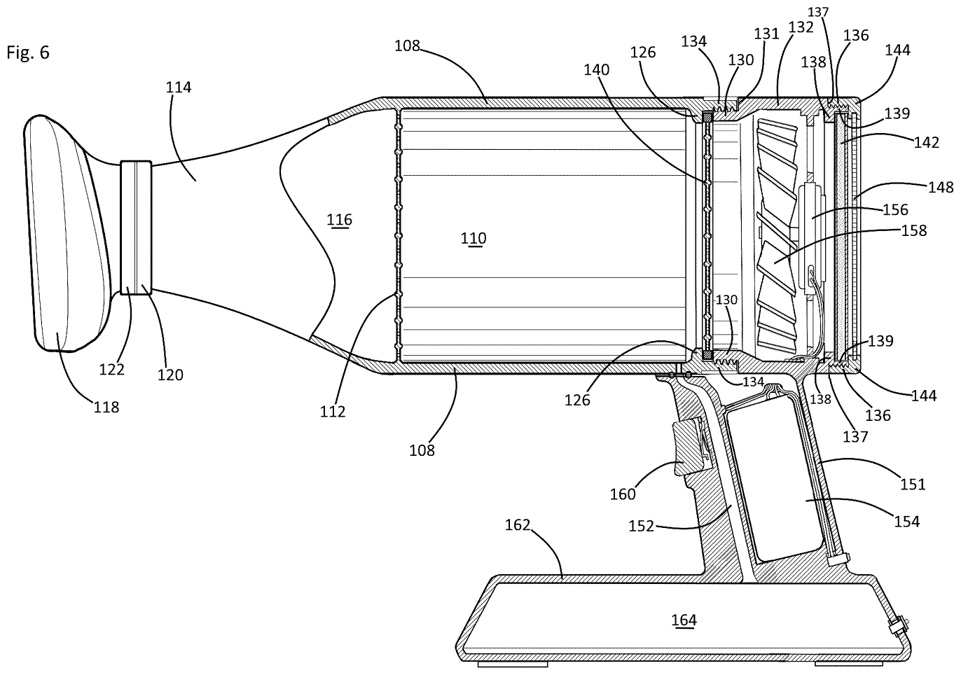

In the alternative configuration of FIGS. 5-8, the instant inventive apparatus for assisting infant respiration is referred to generally by reference arrow 106, such apparatus comprising a cylindrical forward case segment 108 and a cylindrical rearward case segment 132. Rearward and forward ends of such case segments 108 and 132 meet at a circular seam 131 and male and female or external and internal helical threads 130 and 134 releasably secure such segments at such seam.

A forward ice retaining grate or grid 112 is fixedly attached at the forward end of an ice chamber 110 within the forward segment 108, and a removable rearward ice retaining grate or grid 140 is releasably captured by the extreme forward end of the rearward segment 132. Upon screw attachment of the forward segment 108 upon the rearward segment 132, a peripheral flange 126 formed at the rearward end of the forward segment may advantageously capture and hold the rearward grate 140 against the extreme forward peripheral edge of the rearward segment 132. Unscrewing and removing the forward segment 108 from the rearward segment 132 allows chamber 110 to be opened via rearward extraction of the rearward ice retaining grate 140.

In the FIGS. 5-8 configuration, forward components of the case's forward segment 108 including facemask 118, conic section 114, conic space 116, and releasable coupler 120, 122 are configured similarly with similar structures appearing in FIGS. 1-5. Also, in the FIGS. 5-8 configuration, handle 151, base 162, reservoir 164, channel 152, lithium-ion battery 154, fan 158, and motor 156, are configured similarly with similar structures appearing in FIGS. 1-5.

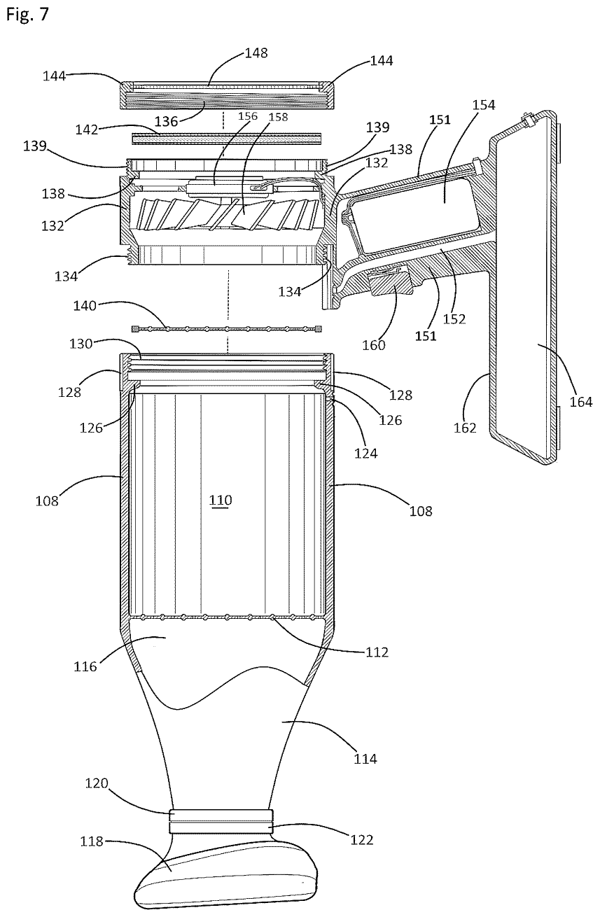

In the FIGS. 5-8 configuration, a circular or disk-shaped HEPA filter 142 may be inserted, removed, and interchanged via screw turning and counterturning of an internally helically threaded retainer sleeve 144. Such sleeve defines a rearward intake port which is covered by screen 148, and presents peripheral internal or female helical threads 136, which are fitted for engagement with male threads 139 presented at the rearward end of the rearward segment 132. While the retainer sleeve 144 is unscrewed and removed as indicated in FIG. 7, HEPA filter 142 may be forwardly inserted into the space peripherally defined by external helical threads 139, such forward HEPA filter extension being stopped by an annular retainer flange 138. Following installation of the HEPA filter 142, the retainer flange 144 may be threadedly re-attached to securely capture such filter at its air filtering use position between the fan 158 and the screen covered air intake port 148. Upon such retainer flange reattachment, the rearward segment 132 meets the flange 144 at seam 137.

In use of the apparatus of FIGS. 5-8, the caretaker may initially vertically orient the apparatus and may grasp the case's forward segment 108 at and about friction ridges 128. The caretaker may then use his or her other hand to apply rotary torque to the case's rearward segment 132, inducing a segment unscrewing rotary motions. In applying such segment unscrewing rotary torque, the handle 151 may advantageously function as a turn assisting lever. Manual disengagement of threads 130 from threads 134 separates the case's segments 108 and 132 as indicated in FIG. 7. Thereafter, the caretaker may remove grate 140 and may pour ice cubes or crushed ice downwardly into space 110, substantially filling such space as indicated in FIG. 8. In the FIG. 8 configuration, the forward segment's underlying forward grate 112 retains the body of ice 113.

Thereafter, the caretaker may downwardly insert the removable grate or grid 140 into the upwardly oriented rear opening of the forward segment 108 until the grate's annular peripheral edge rests upon the upwardly facing annular land formed by retainer flange 126. Thereafter, the operator may threadedly engage the forward segment 108 with the rearward segment 132 until the extreme forward end of the reward segment 132 contacts and compressively holds the grate 140 against retainer flange 126. Accordingly, upon screw threaded reassembly of the case 108, 132, the ice retaining grates 112 and 140 are securely and immovably held at the forward and rearward ends of the ice chamber 110.

Following performance of the ice filling and case reassembly steps indicated in FIG. 8, the caretaker may turn the apparatus 90 degrees, and may place the apparatus upon a flat surface such as the bedside table. Upon operation of the device, air drawn by fan 158 inwardly through intake port 148 is initially cleaned by HEPA filter 142, and is successively cooled and humidified by ice 113 prior to emission at the forward air mask 118 into the infant's mouth and nostrils. During such operation, melt water from the ice 113 advantageously drains downwardly through port 124, and further downwardly through handle channel 152 into the base's reservoir 164.

While the principles of the invention have been made clear in the above illustrative embodiment, those skilled in the art may make modifications to the structure, arrangement, portions, components, and method steps of the invention without departing from those principles. Accordingly, it is intended that the description and drawings be interpreted as illustrative and not in the limiting sense, and that the invention be given a scope commensurate with the appended claims.

I claim:

1. An apparatus for assisting infant respiration comprising:
(a) A segmented case comprising a forward segment, a rearward segment, and a seam between said segments;
(b) Segment mounting means connected operatively to the forward and rearward segments, the segment mounting means being adapted for releasably positioning ends of the forward and rearward segments at the seam;
(c) Output and intake ports respectively opening the case at the forward and rearward segments;
(d) A respirator mask;
(e) Respirator mask mounting means operatively connecting the respirator mask to the air output port;
(f) A fan and electric motor combination mounted within the rearward segment, said combination being adapted for drawing air into the intake port, and for driving the air toward the forward segment; and
(g) A water based air-cooling media within the forward segment.

2. The apparatus for assisting infant respiration of claim 1, wherein the water based air-cooling media comprise ice or an hydrophilic material.

3. The apparatus for assisting infant respiration of claim 2, wherein the segment mounting means comprise a connector selected from the group consisting of releasable latches, pressure fitted joints, and helically threaded joints.

4. The apparatus for assisting infant respiration of claim 3, further comprising a handle having upper and lower ends, the handle being fixedly attached to and extending downwardly from the case's rearward segment.

5. The apparatus for assisting infant respiration of claim 4, wherein the handle is hollow, and further comprising an electric storage battery housed within the handle, the electric storage battery being electrically connected to the fan and electric motor combination's electric motor.

6. The apparatus for assisting infant respiration of claim 3, further comprising an on/off switch connected electrically to the fan and electric motor combination's electric motor.

7. The apparatus for assisting infant respiration of claim 6, wherein the on/off switch is positioned at the handle.

8. The apparatus for assisting infant respiration of claim 4, further comprising a first drain port opening the case at the case's forward segment.

9. The apparatus for assisting infant respiration of claim 8, further comprising a drain channel within the handle, the drain channel having an input end communicating with the first drain port.

10. The apparatus for assisting infant respiration of claim 9, wherein the drain channel has an output end, and further comprising a water reservoir fixedly attached at a lower end of the handle, the drain channel's output end communicating with the water reservoir.

11. The apparatus for assisting infant respiration of claim 10, further comprising a base fixedly attached to the handle's lower end, the base comprising the water reservoir.

12. The apparatus for assisting infant respiration of claim 1, wherein the mask mounting means comprise a releasable coupler.

13. The apparatus for assisting infant respiration of claim 12, wherein the releasable coupler comprises a flexible hose.

14. The apparatus for assisting infant respiration of claim 1, further comprising an air filter fixedly mounted at the intake port.

15. The apparatus for assisting infant respiration of claim 1, further comprising a basket within the casing's forward segment, the air-cooling media being received within the basket.

16. The apparatus for assisting infant respiration of claim 15, wherein the case is opened by an air-cooling media port, said port being positioned at the case's forward segment.

17. The apparatus for assisting infant respiration of claim 16, wherein the air-cooling media port is fitted for insertions and extractions of the basket.

18. The apparatus for assisting infant respiration of claim 17, further comprising a lid, fixedly attached to the case's forward segment, the lid being adapted for alternatively closing and opening the air-cooling media port.

19. The apparatus for assisting infant respiration of claim 1, wherein the case's forward and rearward segments have a circular cross-sectional shape.

20. The apparatus for assisting infant respiration of claim 19, wherein the segment mounting means comprise male and female helical threads positioned at a rearward end of the case's forward segment and at a forward end of the case's rearward segment.
